(19) United States
(12) Patent Application Publication
(10) Pub. No.: US 2004/0248771 A1
(43) Pub. Date: Dec. 9, 2004

(54) Pharmaco-dietary preparation having a nutrition-supplementing and nutrition-enhancing effect

(76) Inventor: Giuseppe Raggi, Lugano (CH)

Correspondence Address: YOUNG & THOMPSON, 745 SOUTH 23RD STREET, 2ND FLOOR, ARLINGTON, VA, 22202, US

(21) Appl. No.: 10/494,286

(22) PCT Filed: Oct. 15, 2002
(86) PCT No.: PCT/IB02/04242

(30) Foreign Application Priority Data
Nov. 1, 2001 (GB) 0126194.0

(51) Int. Cl. A61K 38/00; A61K 31/195

(52) U.S. Cl. 514/2; 514/561

(57) ABSTRACT

A pharmaco-dietary preparation having a nutrition-supplementing and nutrition-enhancing effect and comprising: a) a hydrolysate of amino acids and/or peptides having a relative molecular mass between 10² and 2×10⁴ daltons obtained from proteins; b) β-alanine in an amount equal to, or greater than, 0.1% of the amnioacyl total of the dydrolysate of amino acids and/or peptides.

The present invention relates to pharmaceutical and/or dietary compositions and/or functional human and/or animal foods capable of promoting a reduction of excess weight, preventing aging processes, and assisting in the treatment of disorders linked thereto: atherosclerosis, hypertension, diabetes, osteoporosis, menopausal syndromes, senile cerebral disorders (Alzheimer's disease, Parkinson's disease, dementias and memory losses), psychophysical stresses, depression, chronic fatigue syndrome, cutaneous and dermal aging (wrinkles, cellulitis, alopecia, et cetera), benign prostate hypertrophy, et cetera.

Free radicals (and the peroxidative processes they induce), together with protein malnutrition (often caused by inefficient digestion of proteins and/or by a reduced efficiency of intestinal absorption of amino acids) and with deficits of vitamins, oligoelements and minerals and vitamin-like factors (for example nucleosides derived from the digestion of nucleic acids), have long been recognized as the primary causes of metabolic and structural alterations (such as excess weight, high plasma levels of cholesterol, triglycerides, glucose, reduced levels of antioxidant defences in plasma and in the various tissues, energy deficits of mitochondria and of cell metabolism, damage to DNA and RNAs) that occur in various situations of psychophysical stress and during aging, as well as during the onset of many disorders correlated to aging such as atherosclerosis, diabetes, hypertension, et cetera (Supplement to “The American Journal of Clinical Nutrition”, vol. 53 (No. 1), 1991, p. 189; “Lipid Peroxidation”: part II: “Pathological Implications”, 1987, Chemistry and Physics of Lipids, vol. 45 (no. 2-4), p. 103; “Undernutrition in elderly people”, 1989, Age Ageing, vol. 18, p. 339; “Malnutrition and falls”, 1990, Lancet, vol. 336, p. 1447).

In order to avoid all these pathological degenerations, the inventor of the present invention has devised a preparation that has marked organoleptic virtues. The invention is in fact constituted by a preparation as described in the accompanying claim 1.

A description is now given of some preferred embodiments of the preparation according to the invention, chosen among the many available to a person skilled in the art who follows the teachings contained in the accompanying claim 1.

The composition of the preparation according to the invention essentially comprises:

a hydrolysate of amino acids and/or peptides, with a relative molecular mass between 10² and 2×10⁴ daltons, obtained by hydrolysis of proteins having a high biological value (for example proteins of milk serum, soybean, eggs, wheat, maize, yeasts, fish, meat, et cetera) with the addition of β-alanine in an amount ≧0.1% of the aminoacyl total and preferably between 1 and 3%. Said hydrolysate must receive a further addition of glycine (≧1.5% of the aminoacyl total) and/or glutamine (≧3% of the aminoacyl total and/or taurine (≧0.1% of the amino acyl total) and/or arginine (≧2.1% of the aminoacyl total) if these amino acids are not already present in the above cited amounts.

To boost the effects of the preparation according to the invention it is possible to add:

a hydrolysate of oligonucleotides and/or nucleotides and/or nucleosides, obtained by hydrolysis from ribonucleic and/or deoxyribonucleic acids extracted from yeasts, plants, meat or fish, with a relative molecular mass preferably between 10² and 10⁴, optionally with the addition of adenosine (so that the amount of adenine is ≧10% of the total of all the nitrogen bases present in the oligonucleotides and/or nucleotides and/or nucleosides of the hydrolysate).

a mixture of protein extracts having a hydrolytic activity, of plant and/or animal and/or bacterial origin (for example extracts of Aspergillus oryzae fermented in the presence of rice starch).

a mixture containing D-ribose and/or xylitol.

Furthermore, the preparation according to the invention can contain other components used conventionally, such as for example:

the different species of vitamins and/or vitamin-like products (for example carnitine, creatine, carnosine, homo-carnosine, anserine, betaine, lipoic acid, essential fatty acids of the w-6 and w-3 series, lecithins, inositol, et cetera)

the various species of minerals and oligoelements

carbohydrates of various kinds (glucose, fructose, saccharose, lactose, arabinose, starches, maltodextrins, et cetera)

indigestible fibres and/or polysaccharides (inulins, pectins, celluloses, cyclodextrins, et cetera)

extracts of plants and/or spices and/or medicinal plants containing phytosterols, bioflavones, terpenes, essential oils, et cetera.

As regards the dosage of the various compounds of the preparation, it can be defined within a wide discretionary range: however, the inventor suggests, for the preparation, a dosage of 0.05+5.0 grams of preparation per day per kilogram of body weight of the person taking it, although optimum dosage is between 0.5 and 2.0 grams per day per kilogram of body weight.

As regards the relative dosage of the individual components of the preparation, the inventor suggests to use preferably an amount of said hydrolysate of oligonucleotides and/or nucleotides and/or nucleosides between 1 and 10 mg per day per kg of body weight.

For said mixture of protein extracts having hydrolytic activity of plant and/or animal origin, the inventor suggests a dosage between 0.01 and 2 grams, but preferably between 0.1 and 0.5 grams, per kg of body weight per day.

For said mixture containing D-ribose and/or xylitol, moreover, the inventor suggests a daily dosage in which the administered amount of D-ribose is 0.1 to 250 mg, but preferably 1+25 mg, per kg of body weight, and the amount of xylitol is 0.1 to 1000 mg, but preferably 2 to 100 mg, per kg of body weight. Obviously, the preparation according to the invention can be administered as a single daily dose or split into multiple doses.

The powder and/or granulated forms of the above described components, which are perfectly miscible and usable with each other, are formulated in a composition suitable for oral administration, such as sachets containing powders or granulates; pastilles and dragées; ordinary or effervescent tablets; pasta, rice, crackers, bread, biscuits or other bakery products obtainable by mixing the various active ingredients, in the form of powders and/or granulates, with appropriate food-grade pharmacologically insert excipients, such as simple or complex carbohydrates (food-grade flours of various origin, starches, vegetable fibres of various kinds, and celluloses, chitins or chitosans, pectins, inulins, saccharose, lactose, et cetera); sauces, condiments, creams and/or mayonnaises obtainable by mixing the active ingredients with oils, water, lecithin, natural emulsifiers and any other ingredient normally used in this type of preparation; powdered dispersions for extemporaneous production of milk-beverages and yogurth, beverages of various kinds; appropriately flavoured chewing-gums, et cetera. A preparation according to the invention can also be used in the cosmetic application-field in the form of a cream, of a gel or the like, via aerosol etc.

Two non-limitative examples of possible formulations according to the present invention are presented hereinafter.

### EXAMPLE 1

A) 100 g of a hydrolysate of amino acids and/or peptides (with a relative molecular mass between 10² and 2×10⁴) from milk serum proteins, having the following amino acid composition:

| | | |
|---|---|---|
| Alanine | 5.04 | g |
| Arginine | 2.184 | g |
| Aspartic acid | 10.164 | g |
| Cystine | 3.024 | g |
| Glutamine and | 15.12 | g |
| glutamic acid | | |
| Glycine | 1.512 | g |
| Histidine | 1.764 | g |
| Isoleucine | 4.956 | g |
| Leucine | 12.096 | g |
| Lysine | 9.66 | g |
| Methionine | 2.101 | g |
| Phenylalanine | 3.276 | g |
| Proline | 4.368 | g |
| Serine | 3.024 | g |
| Threonine | 4.452 | g |
| Tryptophan | 2.100 | g |
| Tyrosine | 3.444 | g |
| Valine | 4.704 | g |

with the addition of

4 g glycine

6 g glutamine

1500 mg β-alanine

250 mg taurine

+

B) 2 g of a mixture of oligonucleotides, nucleotides and nucleosides (with a relative molecular mass between 2×10² and 10×10³ daltons) obtained by hydrolysis of nucleic acids from yeast, with the addition of 500 mg of adenosine.

+

C) 8 g of a mixture of protein extracts having a hydrolytic activity (4 g of protein extract of pineapple stalk rich in bromelain+4 g of pancreatic protein extract rich in trypsin, chymotrypsin, et cetera)

+

D) 5 g of a mixture of D-ribose (1 g) and xylitol (4 g).

### EXAMPLE 2

A) 127.25 g of a mixture of protein and nucleotide hydrolysates, protein extracts having proteolytic activity and D-ribose and xylitol as in example 1 A)+1 B)+1 C)+1D)

+

B) a mixture of vitamins, vitamin-like factors, minerals and oligonucleotides, carbohydrates and fibres constituted by:

| | | | | | | | | | | | |
|---|---|---|---|---|---|---|---|---|---|---|---|
| Inulins and | 8 | g | B6 | 8 | mg | Zn | 20 | mg | Phosphor | 200 | mg |
| pectins | | | | | | | | | | | |
| Vitamin A | 2 | mg | B12 | 200 | μg | Cu | 100 | μg | Sodium | 300 | mg |
| Vitamin E | 100 | mg | Biotin | 200 | μg | Boron | 100 | μg | Maltodextrins | 22 | g |
| Vitamin C | 100 | mg | Folic acid | 200 | μg | Cr | 100 | μg | w-6 and w-3 | 4 | g |
| | | | | | | | | | essential fatty | | |
| | | | | | | | | | acids | | |
| Vitamin D | 12 | μg | Inositol | 400 | mg | Vanadium | 40 | μg | Lecithins | 4 | g |
| Vitamin B1 | 4 | mg | Ca | 200 | mg | Molybdenum | 100 | μg | Creatine | 4 | g |
| Vitamin B2 | 4 | mg | Magnesium | 200 | mg | Iodine | 100 | μg | Lipoic acid | 200 | mg |
| B3 | 60 | mg | Potassium | 600 | mg | Iron | 4 | mg | | | |
| nicotinamide | | | | | | | | | | | |
| B5 | 20 | mg | Chloride | 300 | mg | Mn | 10 | mg | | | |
| pantothenic | | | | | | | | | | | |
| acid | | | | | | | | | | | |

In order to study the pharmacological and/or dietary characteristics of the composition according to the present invention, a series of experimental tests on rats and clinical tests in man was conducted.

As regards experimental tests on rats, 60 male rats, divided into 5 groups of 12 animals each, were used. Each group of animals was subjected to a dietary regimen as listed in Table I according to times and methods indicated in Table II.

At the end of the treatments, various body composition parameters were measured (initial and final weight, percentage compositions of H₂O, proteins and fats in the body, variation of levels of deposit of epididymal and perirenal fats; Table III; blood levels of total cholesterol, HDL cholesterol, triglycerides and glucose (Table III): levels of lipoperoxides (MDA) in plasma, liver, brain and heart, hepatic content of reduced glutathione (GSH), and consumption of hepatocellular oxygen and renal levels of 8-oxo-d-guanosine (Table IV).

Experimental data listed in Tables III and IV show that:

Treatment for 84 days with a diet rich in fats with respect to the standard diet induced a dramatic and significant increase in body weight and fat content of the animal, with a decrease in protein masses and in the state of hydration of tissues. There was also a significant increase in blood levels of triglycerides, cholesterol and glucose. Levels of lipoperoxides in plasma and in the various tested tissues were also increased (a clear indicator of reduced efficiency of antioxidant defences!), and there was also a dramatic decrease in liver content of reduced glutathione (further confirmation of the drop in antioxidant defences!). There was also a reduction in the consumption of hepatocellular oxygen (an indicator of reduced energy efficiency of mitochondrial functions!) and a considerable renal increase in 8-oxo-d-guanosine (an indicator of structural and functional damage to nuclear and/or mitochondrial DNA). All these forms of damage indicated a loss of tissue functionality and predisposition to accelerated aging and to the onset of the dysmetabolic disorders correlated thereto (atherosclerosis, diabetes, hypertension, et cetera).

Administration of restricted-calorie diets constituted by milk serum proteins, either untreated (MSP) or hydrolysed (HMSP), was capable of producing a modest preventative effect on the onset of these metabolic-functional alterations.

Administration of restricted-calorie diets constituted by hydrolysates of milk serum protein plus the supplements as listed in example 1 of the present invention (HMSP+I) was instead capable of producing a considerable and surprising synergistic effect in:

facilitating reduction of excess body weight

increasing lean protein mass and decreasing excess accumulated body fat

facilitating tissue hydration

improving antioxidant defences in plasma and in the various tissues

improving the energy-functional efficiency of the mitochondrion

reducing damage and mutations affecting nuclear and/or mitochondrial DNA

These therapeutic benefits, obtainable by administering the protein hydrolysates with the addition of the various supplements claimed in the invention, are always significantly greater than the sum of the benefits obtainable by administering separately the protein fractions alone (untreated or hydrolysed, or as the various individual components of the integrated mixture).

In human clinical tests, several groups of overweight individuals, both healthy and affected by one or more of the many dysmetabolic disorders often correlated to aging and/or excess weight (atherosclerosis, diabetes, hypertension, cerebral-degenerative disorders such as Alzheimer's disease, senile dementias, memory loss, et cetera, osteoporosis and menopausal syndromes, states of psychophysical stress, chronic fatigue syndrome, skin aging, wrinkles, cellulite, alopecia, et cetera, benign prostate hypertrophy, et cetera) were subjected to a dietary treatment with the mixture formulated according to the invention as described in example 2. The doses of the mixture and the administration times varied according to the groups being treated and the extent of the initial excess weight.

In all the clinical studies that were conducted, the beneficial and therapeutic effects obtainable by administering the mixtures formulated according to the invention as listed in example 2 were always been found highly significant in reducing excess weight and improving aging predictive indices, in improving antioxidant defences in plasma (evaluated by monitoring the levels of MDA), in reducing damage to nuclear and/or mitochondrial DNA (evaluated by monitoring 8-oxo-d-guanosine in cells of the mucous membrane of the mouth and/or in urine), and in improving the various tested clinical parameters.

These beneficial aspects observable by administering the mixture formulated completely according to the invention were always been far greater than the effects obtainable by administering the various components (individually or in partial association) that constitute the formulated mixture.

The beneficial and therapeutic effects obtained by administering the mixture formulated according to the present invention also proved themselves capable of increasing and synergistically combining the therapeutic benefits obtainable with the drugs normally used in the various tested disorders; hence the evidence of a possible additional benefit of the use of these supplements: their synergistic effects in assisting the therapeutic action of drugs normally in use in the various disorders cited above.

TABLE I
Percentage composition of experimental diets

| | Obesity- | | Restricted-calorie diets | | |
|---|---|---|---|---|---|
| | inducing diet | | | | HMSP + supplements |
| | Standard | Fat-rich | | | as listed in |
| Components | diet* | diet | MSP | HMSP | Example 1 |
| Standard diet | 100.0 | 60.0 | = | = | = |
| Milk serum proteins (MSP) | | | 40 | | |
| Hydrolysed proteins | | | | 40 | |
| from milk serum | | | | | |
| (HMSP) | | | | | |
| HMSP + supplements | | | | | 50 |
| as listed in Example 1 | | | | | |
| Starch | | | 35.3 | 35.3 | 25.3 |
| Saccharose | | | 10 | 10 | 10 |
| Lard + hydrogenated | | 40 | | | |
| soya oil = (1 + 3) | | | | | |
| Soya oil | | | 5 | 5 | 5 |
| Cellulose | | | 5 | 5 | 5 |
| Mixture of minerals | | | 3.5 | 3.5 | 3.5 |
| Mixture of vitamins | | | 1.0 | 1.0 | 1.0 |
| Choline bitartrate | | | 0.2 | 0.2 | 0.2 |

The standard diet is constituted by: casein (20%); starch (55.3%); saccharose (10%); soya oil (5%); cellulose (5%); mixture of minerals (3.5%); mixture of vitamins (1%); choline bitartrate (0.2%).

TABLE II
Experimental protocol
(day)

| | | | | |
|---|---|---|---|---|
| 0 | 28 | 56 | 84 | 112 |

| | |
|---|---|
| Standard diet (12 rats) | |
| Fat-rich diet (28 rats) | MSP diet |
| | (12 rats) |
| Fat-rich diet (48 rats) | HMSP diet |
| | (12 rats) |
| Fat-rich diet (48 rats) | HMSP + S diet |
| | (12 rats) |

1^{st} group, standard diet: sacrificed on day 84
2^{nd} group, fat-rich diet (FR): sacrificed on day 84
3^{rd} group, FR diet + MSP diet: sacrificed on day 112
4^{th} group, FR diet + HMSP diet: sacrificed on day 112
5^{th} group, FR diet + HMSP + S diet: sacrificed on day 112

TABLE III
Evaluation of the various body composition parameters and blood levels of
glucose, cholesterol and triglycerides in rats subjected
to the various reference diets and
to restricted-calorie diets.

| | | | Restricted-calorie diets | | |
|---|---|---|---|---|---|
| | | | | Hydrolysed | HMSP + supplements |
| | Reference diets | | Serum | milk serum | as listed in |
| | Standard | Fat-rich | proteins | proteins | Example 1 |
| | diet | diet | (MSP) | (HMSP) | (HMSP + S) |
| Initial body weight | 93.0 | 94.4 | 474.4 | 478.6 | 476.4 |
| (g) | | | | | |
| Final body weight | 391.5 | 476.2 | 438.4 | 432.4 | 408.5 |
| (g) | | | | | |
| Daily weight gain | +3.5 | +4.5 | −1.2 | −1.6 | −2.4 |
| or loss (g/day) | | | | | |
| Epididymal fat | 2.16 | 3.44 | 3.22 | 2.98 | 2.61 |
| content (g/100 g of | | | | | |
| body weight) | | | | | |
| Perirenal fat content | 2.61 | 4.42 | 3.90 | 3.75 | 3.02 |
| (g/100 g of body | | | | | |
| weight) | | | | | |
| % H₂O content of | 58.4 | 51.2 | 52.6 | 53.5 | 56.0 |
| carcasses | | | | | |
| % protein content | 19.4 | 16.9 | 17.6 | 18.1 | 19.1 |
| of carcasses | | | | | |
| % fat content of | 18.0 | 27.4 | 25.1 | 23.6 | 19.7 |
| carcasses | | | | | |
| Blood glucose | 9.87 | 10.54 | 11.26 | 10.68 | 9.21 |
| (mmol/liter) | | | | | |
| Blood triglycerides | 1.07 | 1.54 | 1.39 | 1.17 | 0.86 |
| (mmol/liter) | | | | | |
| Total blood | 1.68 | 1.94 | 2.24 | 2.12 | 1.78 |
| cholesterol | | | | | |
| (mmol/liter) | | | | | |
| Blood HDL | 0.99 | 1.07 | 1.24 | 1.15 | 1.05 |
| cholesterol | | | | | |
| (mmol/liter) | | | | | |

TABLE IV
Levels of lipoperoxides as nmols of malonyldialdehyde (MDA) per g of tissue or
per ml of plasma, variations in hepatocellular oxygen consumption and
hepatic levels of reduced glutathione (GSH), and variations in kidney
levels of 8-oxo-d-guanosine in rats subjected to the various diets.

| | Restricted-calorie diets | | | | |
|---|---|---|---|---|---|
| | Reference diets | | | Hydrolysed | HMSP + supplements |
| | | Fat- | Milk serum | milk serum | as listed in |
| | Standard | rich | proteins | proteins | example I |
| | diet | diet | (MSP) | (HMSP) | (HMSP + I) |
| *plasma MDA | 2.5 | 5.1 | 5.0 | 4.6 | 3.4 |
| *liver MDA | 25.6 | 44.8 | 41.5 | 36.8 | 28.9 |
| *brain MDA | 55.4 | 108.5 | 102.4 | 98.5 | 76.5 |
| *heart MDA | 24.8 | 45.6 | 40.2 | 36.8 | 29.8 |
| **hepatocellular | 276.4 | 194.5 | 206.8 | 228.4 | 259.3 |
| oxygen | | | | | |
| consumption μmols | | | | | |
| O₂/min per 10⁷ | | | | | |
| cells) | | | | | |
| **hepatic GSH | 36.1 | 22.4 | 28.0 | 32.2 | 40.8 |
| nmols/10⁵ cells | | | | | |
| ***Kidney levels of | 2.87 | 3.65 | 3.56 | 3.48 | 3.08 |
| 8-oxo-7,8 dehydro- | | | | | |
| 2′-deoxyguanosine | | | | | |
| expressed as ratio | | | | | |
| with respect to d- | | | | | |
| guanosine (×10⁵) | | | | | |

*MDA is dosed according to the method of K. Yogi et al., 1982 “Lipid Peroxides in Biology and Medicine”, Academic Press, New York, pages 324-340
**O₂ consumption and levels of GSH in liver are dosed according to the method of T. M. Hagen et al., 1999, FASEB J., vol. 13, pages 411-418
***the kidney level of 8-oxo-d-guanosine is dosed according to the method of M. Karbownik et al., 2001, Mutation Res., 474, pages 87-92.

1-8. (canceled).

9. A pharmaco-dietary preparation having a nutrition-supplementing and nutrition-enhancing effect, comprising:
a hydrolysate of amino acids and/or peptides having a relative molecular mass between 10² and 2×10⁴ daltons obtained from proteins, and β-alanine in an amount equal to, or greater than, 0.1% of the aminoacyl total of said hyudrolysate of amino acids and/or peptides,
a hydrolysate of oligonucleotides and/or nucleotides and/or nucleosides obtained by hydrolysis from ribonucleic and/or deoxyribonucleic acids extracted from yeast, plants, meat or fish, having a relative molecular mass between 10² and 10⁴, optionally with the addition of adenosine so that the amount of adenine is ≧10% of the total of all nitrogenous bases present in the oligonucleotides and/or nucleotides and/or nucleosides of the hydrolysate, and/or
a mixture of protein extracts having a hydrolytic activity of plant and/or animal and/or bacterial origin, and/or
a mixture containing d-ribose and/or xylitol.

10. The preparation according to claim 9, further comprising glycine in an amount equal to, or greater than, 1.5% of the aminoacyl total of said hydrolysate of amino acids and/or peptides.

11. The preparation according to claim 9, further comprising glutamine in an amount equal to, or greater than, 3% of the aminoacyl total of said hydrolysate of amino acids and/or peptides.

12. The preparation according to claim 9, further comprising taurine in an amount equal to, or greater than, 0.1% of the aminoacyl total of said hydrolysate of amino acids and/or peptides.

13. The preparation according to claim 9, further comprising arginine in an amount equal to, or greater than, 2.1% of the aminoacyl total of said hydrolysate of amino acids and/or peptides.

14. The preparation according to claim 9, further comprising:
a hydrolysate of oligonucleotides and/or nucleotides and/or nucleosides obtained by hydrolysis from ribonucleic and/or deoxyribonucleic acids extracted from yeasts, plants, meat or fish, having a relative molecular mass between 10² and 10⁴, with the addition of adenosine so that the amount of adenine is ≧10% of the total of all nitrogenous bases present in the oligonucleotides and/or nucleotides and/or nucleosides of the hydrolysate, and
a mixture of protein extracts having a hydrolytic activity of at least one of plant, animal and bacterial origin, and
a mixture containing one of d-ribose and xylitol.

15. The preparation according to claim 9, having the consistency of powder or granulate.

16. The preparation according to claim 9, packaged in the form of tablets.
